(19) United States
(12) Patent Application Publication
(10) Pub. No.: US 2012/0123094 A1
(43) Pub. Date: May 17, 2012

(54) GREENER METHOD FOR THE PRODUCTION OF COPOLYMER 1

(75) Inventor: Ettigounder Ponnusamy, St. Louis, MO (US)

(73) Assignee: SIGMA-ALDRICH CO. LLC., St. Louis, MO (US)

(21) Appl. No.: 13/295,183

(22) Filed: Nov. 14, 2011

Related U.S. Application Data
(60) Provisional application No. 61/414,618, filed on Nov. 17, 2010.

(51) Int. Cl. C07K 1/06

(52) U.S. Cl. 530/350

(57) ABSTRACT

The present invention provides a greener method for the production of polyamino acid random copolymers containing alanine, glutamic acid, lysine and tyrosine. In particular, the present invention provides a greener method for the production of Copolymer 1 or a pharmaceutically acceptable salt thereof via a synthetic route that only requires a single deprotection step.

### CROSS REFERENCE TO RELATED APPLICATIONS

This application claims the priority of U.S. provisional application No. 61/414,618, filed Nov. 17, 2011, which is hereby incorporated by reference in its entirety.

### FIELD OF THE INVENTION

The invention provides a method for the production of polyamino acid random copolymers. In particular, the invention provides a greener method for the production of poly (alanine, glutamic acid, lysine, tyrosine) or a pharmaceutically acceptable salt thereof via a synthetic route that only requires a single deprotection step.

### BACKGROUND OF THE INVENTION

Polyamino acid random copolymers have a wide variety of properties that mimic proteins. These properties make polyamino acid random copolymers suitable for the treatment of certain diseases. For example, polyamino acid random copolymers comprising alanine, glutamic acid, lysine, and tyrosine have been used in the treatment of Multiple Sclerosis (MS). Poly (L-alanine, L-glutamic acid, L-lysine, L-tyrosine) where the polypeptides contain L-alanine, L-glutamic acid, L-lysine, and L-tyrosine in a ratio of 6:2:5:1, respectively, is sold as COPAXONE®, and is used to treat MS.

Synthesis of poly (alanine, glutamic acid, lysine, tyrosine) requires protecting groups for the side chains of the glutamic acid and lysine residues. The manufacture of poly (alanine, glutamic acid, lysine, tyrosine) copolymer is typically achieved by protecting lysine with an N^{ε}-TFA protecting group and protecting glutamic acid with a γ-benzyl protecting group. Removal of the TFA and benzyl protecting groups requires two separate steps. Removal of the benzyl protecting group from glutamic acid requires using hazardous chemicals (HBr/acetic acid) and highly flammable solvent (acetone or ethyl ether). Also, benzyl bromide, a very strong lachrymator, is generated as a byproduct. To remove the TFA protecting group from lysine, the product needs to be further treated with piperidine, which is also flammable. Significantly, these methods of deprotection require long reaction times and result in polyamino acid random copolymers in a reduced yield and with variable molecular weights. Therefore, a need exists for an efficient and less toxic process for the manufacture of polyamino acid random copolymers containing alanine, glutamic acid, lysine and tyrosine to yield a greener synthetic route than currently exists in the art.

### SUMMARY OF THE INVENTION

Briefly, therefore, the present invention provides a greener method for the synthesis of polyamino acid random copolymers by polymerizing a mixture of N-carboxyanhydride of alanine, N-carboxyanhydride of tyrosine, N-carboxyanhydride of R¹-protected glutamic acid, N-carboxyanhydride of base-labile protected L-lysine in the presence of a polymerization initiator to form a protected polyamino acid random copolymer, then adding a base to the protected polyamino acid random copolymer to cleave both the R¹ group from the glutamic acid residue and the protecting group from the lysine residue.

Other aspects and iterations of the invention are described in more detail below.

### DETAILED DESCRIPTION OF THE INVENTION

A method for the production of polyamino acid random copolymers has been developed. According to one aspect, methods of the syntheses of various stereoisomeric forms of poly (alanine, glutamic acid, lysine, tyrosine) are provided, such as a synthesis of poly (L-alanine, L-glutamic acid, L-lysine, L-tyrosine), known as COPAXONE®. The method is generally faster, more efficient, and uses less toxic reagents than current methods described in the art. As illustrated in the examples, the method is greener in part because it utilizes a single deprotection step for the removal of the protecting groups on the glutamic acid residue and the lysine residue, and eliminates the generation of hazardous waste during the deprotection step. Further, the present methods generally provide polyamino acid random copolymers in improved yields and in a less labor and equipment intensive fashion in comparison to contemporary methods.

(I) Preparation of a Polymerization Reaction Mixture and Polymerization

As is commonly known in the art, polyamino acid copolymers are prepared from N-carboxyanhydride derivatives of amino acids (NCAs). The preparation of NCAs is known to those skilled in the art, and is described in detail in Goodman and Peggion, Pure and Applied Chemistry, volume 53, p. 699, 1981, which is incorporated herein by reference in its entirety and for all purposes as if fully set forth herein. In essence, the amino acid is treated with phosgene in an ethereal solvent such as tetrahydrofuran, to produce the corresponding NCA. The amino acids may be D- or L-amino acid optical isomers. In an exemplary iteration, the amino acids are L-amino acids.

In the present invention, the reaction mixture is comprised of N-carboxyanhydride alanine, N-carboxyanhydride of a R¹-protected glutamic acid, the N-carboxyanhydride of base-labile protected lysine, and N-carboxyanhydride of tyrosine. In this regard, the reaction mixture may be comprised of L optical isomers, D optical isomers, or a mixture of L and D optical isomers of any or all of the foregoing N-carboxyanhydride amino acids. In some embodiments, the reaction mixture comprises N-carboxyanhydride L-alanine, N-carboxyanhydride of a R¹-protected L-glutamic acid, the N-carboxyanhydride of base-labile protected L-lysine, and N-carboxyanhydride of L-tyrosine. In other embodiments, the reaction mixture is comprised of N-carboxyanhydride D-alanine, N-carboxyanhydride of a R¹-protected D-glutamic acid, the N-carboxyanhydride of base-labile protected D-lysine, and N-carboxyanhydride of D-tyrosine. As will be appreciated by a skilled artisan, the molar ratio of the various NCAs in the reaction mixture will directly influence the ratio of each amino acid in the resulting polyamino acid random copolymer. Thus, to achieve a particular molar ratio of amino acid residues in the final polypeptide, the NCAs are added to the reaction mixture in the desired ratio. In one iteration, the NCAs of alanine, glutamic acid, lysine and tyrosine are present in the polymerization reaction mixture in a ratio of about 5:1:4:1 to about 7:3:6:1, respectively. In another iteration, the ratio is 6:2:5:1. In yet another iteration, the ratio of NCAs can vary from 6:2:5:1 by ±20% without departing from the scope of the invention.

The R¹ protecting group for the carboxylate group of the glutamic acid side chain may be a lower, non-aromatic alkyl group containing from one to six carbon atoms in the principle chain. The alkyl group may be straight, branched, or cyclic. In some embodiments, R¹ may be methyl, ethyl, n-propyl, isopropyl, n-butyl, isobutyl, tert-butyl, straight pentyl and branched pentyl. In a preferred embodiment R¹ is selected from the group consisting of methyl and ethyl. In an exemplary embodiment, R¹ is an ethyl.

The protecting group for the amino (ammonium) group of the lysine side chain is a base labile protecting group. Suitable base labile protecting groups for lysine include, but are not limited to, 9-fluorenylmethloxycarbonyl (Fmoc) and trifluoroacetyl (TFA). In an exemplary embodiment, the protecting group is TFA.

The polymerization initiator of the reaction mixture is a nucleophile. The choice of nucleophile can and will vary, and more than one nucleophile may be used. In some embodiments, the nucleophile is selected from the group consisting of amines and metal alkoxides. In some embodiments, the nucleophile is an amine. In one embodiment, the nucleophile is a primary amine. In another embodiment, the nucleophile is a secondary amine. In yet another embodiment, the nucleophile is a tertiary amine. Suitable examples of amine nucleophiles include, but are not limited to, diethylamine, triethylamine, hexylamine, phenylamine, ethylamine, N,N-diisopropylamine, and N,N-dicyclohexylamine. In other embodiments, the nucleophile is a metal alkoxide. Suitable examples of metal alkoxides include metal alkoxides having the formula MOR wherein, M is a metal and R is an alkyl group. In some alternatives of the embodiment, the metal of the metal alkoxide may be an alkali metal such as sodium or potassium, and the alkyl residue may be a linear, branched or cyclic alkyl group having 1 to 10 carbon atoms. Suitable examples of metal oxide polymerization initiators include, but are not limited to, sodium methoxide, sodium ethoxide, sodium propoxide or combinations thereof. In an exemplary embodiment, the metal alkoxide polymerization initiator is sodium methoxide.

As will be appreciated by those of skill in the art, the molar ratio of the NCAs to polymerization initiator used to form the polymerization reaction mixture can and will vary over a wide range, as the molar ratio of the NCAs to the polymerization initiator used to form the polymerization reaction mixture influences the average molecular weight of resulting polyamino acid random copolymer. In general, the average molecular weight of the resulting polyamino acid tends to decrease as the ratio of the NCAs to initiator decreases. For example, to prepare polyamino acid polymers having an average molecular weight in excess of 10,000, it is generally preferred that the molar ratio of the NCAs to the initiator be about 15:1, about 20:1, about 25:1, or a range between and including any two of these values. In one embodiment, the molar ratio of the NCAs to the initiator is in the range of about 15:1 to about 25:1, respectively. The molar ratio of the NCAs comprising the reaction mixture to the polymerization initiator may be about 5:1, about 10:1, about 20:1, about 30:1, about 40:1, about 50:1, about 75:1, about 100:1, about 200:1, about 300:1, about 400:1, about 500:1, about 600:1, about 700:1, about 800:1, about 900:1, about 1,000:1, or a range between and including any two of these values. In one embodiment of the invention, the molar ratio of the NCAs comprising the reaction mixture to polymerization initiator may be about 5:1 to about 1,000:1. In another alternative of the embodiment, the molar ratio of the NCAs comprising the reaction mixture to polymerization initiator may be about 10:1 to about 100:1. In yet another alternative embodiment, the molar ratio of the NCAs to polymerization initiator may be about 100:1 to about 700:1.

The solvent used in the polymerization reaction mixture is typically an organic solvent, or a combination of organic solvents, any or all of which may be an aprotic solvent. Non-limiting examples of suitable aprotic solvents include, but are not limited to, acetone, acetonitrile, diethoxymethane, N,N-dimethylformamide (DMF), dimethyl sulfoxide (DMSO), N,N-dimethylpropionamide, 1,3-dimethyl-3,4,5,6-tetrahydro-2(1H)-pyrimidinone (DMPU), 1,3-dimethyl-2-imidazolidinone (DMI), 1,2-dimethoxyethane (DME), dimethoxymethane, bis(2-methoxyethyl) ether, N,N-dimethylacetamide (DMAC), 1,4-dioxane, N-methyl-2-pyrrolidinone (NMP), ethyl acetate, ethyl formate, ethyl methyl ketone, formamide, hexachloroacetone, hexamethylphosphoramide, methyl acetate, N-methylacetamide, N-methylformamide, methylene chloride, nitrobenzene, nitromethane, propionitrile, sulfolane, tetramethylurea, tetrahydrofuran (THF), 2-methyltetrahydrofuran, toluene, trichloromethane, and combinations thereof. In addition to the above, suitable organic solvents also include, but are not limited to, alkane and substituted alkane solvents (including cycloalkanes), aromatic hydrocarbons, esters, ethers, ketones, combinations thereof, and the like. Specific organic solvents that may be employed, include, for example, acetonitrile, benzene, butyl acetate, t-butyl methyl ether, t-butyl methyl ketone, chlorobenzene, chloroform, chloromethane, cyclohexane, dichloromethane, dichloroethane, diethyl ether, ethyl acetate, diethylene glycol, fluorobenzene, heptane, hexane, isobutyl methyl ketone, isopropyl acetate, methyl ethyl ketone, 2-methyltetrahydrofuran, pentyl acetate, n-propyl acetate, tetrahydrofuran, toluene, and combinations thereof. In one embodiment, the solvent is selected from the group consisting of 1,4-dioxane, chloroform, dichloromethane, acetonitrile, and combinations thereof. In an exemplary embodiment, the solvent used in the polymerization reaction mixture is 1,4-dioxane.

Polymerization of the NCAs may be carried out over a range of temperatures and times without departing from the scope of the invention. By way of non-limiting example, polymerization may be carried out for a period of about 12 hours, about 18 hours, about 24 hours, about 30 hours, or at a range between and including any two of these values. In some embodiments, the polymerization is carried out for a period of about 12 hours to about 30 hours. In exemplary embodiments, the polymerization is carried out for a period of about 18 hours to 24 hours. Similarly, the polymerization may be performed at a variety of temperatures, such as a temperature of about 20° C., about 25° C., about 30° C., about 35° C., about 40° C., or at a range between and including any two of these values. In some embodiments, the polymerization is performed at a temperature of about 20° C. to about 40° C., more typically about 25° C. to about 30° C.

The resulting polyamino acid random copolymer will be poly (alanine, R¹-protected glutamic acid, base-labile protected lysine, tyrosine) where alanine, protected glutamic acid, protected lysine, and tyrosine are incorporated into the polymer in a ratio of about 5:1:4:1 to about 7:3:6:1, respectively. In another iteration, the ratio is 6:2:5:1. In still another iteration, the ratio is 6:2:5:1±20%, respectively. In an exemplary embodiment, the resulting polyamino acid random copolymer will be poly (L-alanine, γ-ethyl L-glutamic acid, N^{ε}-TFA-L-lysine, L-tyrosine) where the L-alanine, γ-ethyl L-glutamic acid, N^{ε}-TFA-L-lysine, and L-tyrosine are in a ratio of (6:2:5:1)±20%, respectively.

The average molecular weight of the polyamino acid random copolymer can vary over a range depending on the number of repeat units in the polymer. In some embodiments, the number of repeat units varies from about 10 to about 1,000, and the polyamino acid random copolymer has a mass average molecular weight from 2,000-100,000. In preferred embodiments, the polyamino acid copolymer has a mass-average molecular weight of about 5,000 to about 10,000.

Upon completion of polymerization, the polyamino acid may be isolated by any number of methods commonly understood in the art. In some preferred embodiments, the polyamino acid is precipitated in water and filtered.

(II) Deprotection Via a Single Hydrolysis Reaction

Advantageously, the R¹ protecting group on glutamic acid and the base-labile protecting group on lysine may be deprotected via a single hydrolysis reaction. The resulting polyamino acid random copolymer will preferably be poly (alanine, glutamic acid, lysine, tyrosine). In an exemplary embodiment the resulting polyamino acid random copolymer will be poly (L-alanine, L-glutamic acid, L-lysine, L-tyrosine).

The hydrolysis reaction may be carried out in the presence of an organic or inorganic base. Suitable bases include, but are not limited to, potassium hydroxide, barium hydroxide, cesium hydroxide, sodium hydroxide, strontium hydroxide, calcium hydroxide, lithium hydroxide, and rubidium hydroxide, cyclohexamine, 1,5-diazabicyclo[5.4.0]undecene, piperidine, ethanolamine, pyrrolidine, diethylamine, morpholine, piperazine, dicycloheylamine, hydroxylamine, N,N′-isopropylamine, tributlyamine, triethylenediamine, monoethanolamine, diethanolamine, and triethanolamine. In some embodiments, the hydrolysis is carried out in the presence of a base selected from the group consisting of sodium hydroxide, potassium hydroxide, rubidium hydroxide, cyclohexamine, 1,5-diazabicyclo[5.4.0]undecene, piperidine, ethanolamine, pyrrolidine, diethylamine, morpholine, piperazine, dicycloheylamine, hydroxylamine, N,N′-isopropylamine, tributlyamine, triethylenediamine, monoethanolamine, diethanolamine, and triethanolamine.

In general, the amount of base added to the protected polyamino acid random copolymer is equal to or in excess of the molar amount of protected groups in the polypeptide. The amount of base to protected groups can vary over a wide range, such as from about 1:1, about 5:1, about 7:1, about 10:1, about 15:1, about 20:1, or about 25:1. In a preferred embodiment, the molar ratio of base to protected groups is from about 5:1 to about 7:1.

In one embodiment, the solvent used in the hydrolysis reaction is a protic solvent. Suitable examples of protic solvents include, but are not limited to, methanol, ethanol, isopropanol, n-propanol, isobutanol, n-butanol, sec-butanol, t-butanol, formic acid, acetic acid, water, and combinations thereof. In an exemplary alternative of the embodiment, the solvent used for the hydrolysis reaction is ethanol.

The hydrolysis reaction may be carried out over a range of temperatures and times without departing from the scope of the invention. By way of non-limiting example, hydrolysis may be carried out for a period of about 20 minutes, about 0.5 hours, about 1 hour, about 2 hours, about 5 hours, or about 10 hours. In some embodiments, hydrolysis may be carried out for a period of about 0.5 hours to about 2 hours. By way of non-limiting example, hydrolysis may be carried out at a temperature of about 15° C., about 20° C., about 25° C., about 30° C., or at a range between and including any two of these values. In some embodiments, hydrolysis may be carried out at a temperature ranging from about 15° C. to about 30° C.

After deprotection of the polyamino acid random copolymers, the product may be optionally purified by any number of methods commonly known in the art. In some embodiments, the product is diluted with water, dialyzed and lyophilized to yield the polyamino acid random copolymer in purified form.

In a further embodiment, the polyamino acid random copolymer is obtained in a salt form. Generally, the salt is a pharmaceutically acceptable salt. The term “pharmaceutically-acceptable salts” are salts commonly used to form alkali metal salts and to form addition salts of free acids or free bases. The nature of the salt may vary, provided that it is pharmaceutically acceptable. Suitable pharmaceutically acceptable acid addition salts of compounds for use in the present methods may be prepared from an inorganic acid or from an organic acid. Examples of such inorganic acids are hydrochloric, hydrobromic, hydroiodic, nitric, carbonic, sulfuric and phosphoric acid. Appropriate organic acids may be selected from aliphatic, cycloaliphatic, aromatic, araliphatic, heterocyclic, carboxylic and sulfonic classes of organic acids, examples of which are formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, 4-hydroxybenzoic, phenylacetic, mandelic, embonic (pamoic), methanesulfonic, ethanesulfonic, benzenesulfonic, pantothenic, 2-hydroxyethanesulfonic, toluenesulfonic, sulfanilic, cyclohexylaminosulfonic, stearic, algenic, hydroxybutyric, salicylic, galactaric and galacturonic acid. Suitable pharmaceutically-acceptable base addition salts of compounds of use in the present methods include metallic salts made from aluminum, calcium, lithium, magnesium, potassium, sodium and zinc or organic salts made from N,N′-dibenzylethylenediamine, chloroprocaine, choline, diethanolamine, ethylenediamine, meglumine-(N-methylglucamine) and procaine. All of these salts may be prepared by conventional means from the corresponding polyamino acid copolymer by reacting, for example, the appropriate acid or base with the polyamino acid copolymer. In a preferred embodiment, the pharmaceutically acceptable salt is acetate.

The copolymer may be analyzed by proton nuclear magnetic resonance (NMR), and the molecular weight determined by gel permeation chromatography multi-angle laser light scattering (GPC MALLS). In one iteration, the yield of poly (L-alanine, L-glutamic acid, L-lysine, L-tyrosine) produced via the method of the invention is at least 60%. In another iteration, the yield is at least 65%. In an exemplary iteration, the yield is greater than 70%. Poly (L-alanine, L-glutamic acid, L-lysine, L-tyrosine) produced via the method of the invention also has a high degree of optical purity. For example, the poly (L-alanine, L-glutamic acid, L-lysine, L-tyrosine) is typically greater than 99% optically pure. In an exemplary iteration, the optical purity is greater than 99.5%.

DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“N-carboxyanhydride” is art-recognized refers to a cyclic amino acid derivative which may be synthesized by a variety of methods including but not limited to the reaction of an amino acid or derivative thereof with phosgene, a phosgene equivalent (such as di- or triphosgene), phosphorous pentachloride, phosphorus tribromide, thionyl chloride, or other suitable reagents.

As used herein, COPAXONE®, glatiramer acetate, Copolymer 1, Cop-1 are used interchangeably.

### EXAMPLES

The following examples specify the synthesis of poly (alanine, glutamic acid, lysine, tyrosine) using N-carboxyanhydrides of L-alanine, γ-ethyl-L-glutamic acid, N^{ε}-trifluoroacetyl-L-lysine and L-tyrosine. Other stereoisomeric forms of poly (alanine, glutamic acid, lysine, tyrosine) may be readily prepared with minor adaptations of the procedures described in the examples herein, such as through the use of D- or D/L- forms of any or all of the indicated N-carboxyanhydrides.

The techniques used for the polymerization of the N-carboxyanhydrides (NCA) to polymers are known to those skilled in the art and are given in detail in the review article by M. Goodman and E. Peggion, Pure and Applied Chemistry, volume 53, p. 699, 1981 and the book by H. R. Kricheldorf “Alpha amino acids-N-Carboxyanhydrides and Related Heterocycles”, Springer Verlag (1987) and also the recent publications by Wendelmoed N. E. van Dijk-Wolthuis et al, Macromol. Chem. Phys. Volume 198, p. 3893-3906, 1997.

### Example 1

Polymerization: 3.334 g (0.028997 mole) of L-alanine NCA, 1.942 g (0.009662 mole) of γ-ethyl-L-glutamic acid NCA, 6.479g (0.024157 mole) of N^{ε}-TFA-L-lysine NCA and 1.000 g (0.004831 mole) of L-tyrosine NCA were dissolved in 0.225 liter of 1,4-dioxane to make a 0.3M solution. Added ˜1.25 g of charcoal and filtered to get clear colorless solution. The filtered NCA solution was transferred to a 1 liter three neck RB flask equipped with mechanical mixing and a water bath at a temperature of 25-30° C. 2.7 ml of 1N sodium methoxide (0.0027 moles) was placed in 25 ml of 1,4-dioxane. The sodium methoxide solution was added to the NCA solution in one portion with vigorous mixing. The polymerization reaction mixture was mixed for 2 hours and held at 25-30° C. for 18-24 hours.

Precipitation of Protected Polymer: Slowly poured the polymer solution in 1,250 ml of DI-water with vigorous mixing. Protected polymer precipitated, mixed for 30 minutes and filtered and washed the polymer with 5×125 ml of DI-water.

Deprotection of Ethyl and TFA Groups: Transferred the wet polymer to a 500 ml Erlenmeyer flask. Added 140 ml of 1.5M ethanolic sodium hydroxide and mixed for 0.5 hour. Polymer went into solution within 5 minutes of adding the reagent. Diluted the polymer solution with ˜150 ml of DI-water and mixed for 5 minutes.

Dialysis and Lyophilization (Freeze Drying): The poly (alanine, glutamic acid, lysine, tyrosine) sodium solution was dialyzed (18-24 hours) against running deionized water using ˜12K molecular weight cut off dialysis tubing to remove the oligomers and salts. The dialysis tubing was transferred to 3.5% acetic acid solution (˜18 L) and let stand for 7 hours and slowly mix the solution containing the dialysis tubings to complete salt exchange. Then the solution was dialyzed against running de-ionized water for 18-24 hours, collected, filtered through a 0.2-micron filter and lyophilized (freeze dried) to get the solid poly (alanine, glutamic acid, lysine, tyrosine) acetate copolymer.

Yield: 6.21 g (71.9%). Proton NMR showed the complete removal of the ethyl and TFA groups. Measured specific viscosity in water, 1% solution at 25° C. and calculated the viscosity molecular weight (18,000). Also measured GPC MALLS molecular weight (16,800), optical rotation)(−63.2°), and amino acid analysis (alanine:5.70; glutamic acid:1.90; lysine:4.80 and tyrosine:1.00).

### Example 2

Polymerization: 6.667 g (0.057974 mole) of L-alanine NCA, 3.884 g (0.019323 mole) of γ-ethyl-L-glutamic acid NCA, 12.957 g (0.048311 mole) of N^{ε}-TFA-L-lysine NCA and 2.000 g (0.009662 mole) of L-tyrosine NCA were dissolved in 0.45 liter of 1,4-dioxane to make a 0.3M solution. Added ˜2.5 g of charcoal and filtered to get clear colorless solution. The filtered NCA solution was transferred to a 1 liter three neck RB flask equipped with mechanical mixing and a water bath at a temperature of 25-30° C. 5.4 ml of 1N sodium methoxide (0.0054 moles) was placed in 25 ml of 1,4-dioxane. The sodium methoxide solution was added to the NCA solution in one portion with vigorous mixing. The polymerization reaction mixture was mixed for 2 hours and held at 25-30° C. for 18-24 hours.

Precipitation of Protected Polymer: Slowly poured the polymer solution in 2,500 ml of DI-water with vigorous mixing. Protected polymer precipitated, mixed for 30 minutes and filtered and washed the polymer with 5×250 ml of DI-water.

Deprotection of Ethyl and TFA Groups: Transferred the wet polymer to a 1,000 ml Erlenmeyer flask. Added 280 ml of 1.5M ethanolic sodium hydroxide and mixed for 0.5 hour. Polymer went into solution within 5 minutes of adding the reagent. Diluted the polymer solution with ˜300 ml of DI-water and mixed for 5 minutes.

Dialysis and Lyophilization (Freeze Drying): The poly (alanine, glutamic acid, lysine, tyrosine) sodium solution was dialyzed (18-24 hours) against running deionized water using ˜12K molecular weight cut off dialysis tubing to remove the oligomers and salts. The dialysis tubing was transferred to 3.5% acetic acid solution (˜18 L) and let stand for 7 hours and slowly mix the solution containing the dialysis tubings to complete salt exchange. Then the solution was dialyzed against running de-ionized water for 18-24 hours, collected, filtered through a 0.2-micron filter and lyophilized (freeze dried) to get the solid poly (alanine, glutamic acid, lysine, tyrosine) acetate copolymer.

Yield: 12.23 g (70.8%). Proton NMR showed the complete removal of the ethyl and TFA groups. Measured specific viscosity in water, 1% solution at 25° C. and calculated the viscosity molecular weight (17,700). Also measured GPC MALLS molecular weight (16,950), optical rotation)(−62.4°), and amino acid analysis (alanine:6.20; glutamic acid:2.10; lysine:5.10 and tyrosine:1.00).

### Example 3

Polymerization: 6.667 g (0.057974 mole) of L-alanine NCA, 3.884 g (0.019323 mole) of γ-ethyl-L-glutamic acid NCA, 12.957 g (0.048311 mole) of N^{ε}-TFA-L-lysine NCA and 2.000 g (0.009662 mole) of L-tyrosine NCA were dissolved in 0.45 liter of 1,4-dioxane to make a 0.3M solution. Added ˜2.5 g of charcoal and filtered to get clear colorless solution. The filtered NCA solution was transferred to a 1 liter three neck RB flask equipped with mechanical mixing and a water bath at a temperature of 25-30° C. 3.9 ml of 1N sodium methoxide (0.0039 moles) was placed in 25 ml of 1,4-dioxane. The sodium methoxide solution was added to the NCA solution in one portion with vigorous mixing. The polymerization reaction mixture was mixed for 2 hours and held at 25-30° C. for 18-24 hours.

Precipitation of Protected Polymer: Slowly poured the polymer solution in ˜2,500 ml of DI-water with vigorous mixing. Protected polymer precipitated, mixed for 30 minutes and filtered and washed the polymer with 5×250 ml of DI-water.

Deprotection of Ethyl and TFA groups: Transferred the wet polymer to a 1,000 ml Erlenmeyer flask. Added 280 ml of 1.5M ethanolic sodium hydroxide and mixed for 0.5 hour. Polymer went into solution within 5 minutes of adding the reagent. Diluted the polymer solution with ˜300 ml of DI-water and mixed for 5 minutes.

Dialysis and Lyophilization (freeze drying): The poly(alanine, glutamic acid, lysine, tyrosine) Sodium solution was dialyzed (18-24 hours) against running deionized water using ˜12K molecular weight cut off dialysis tubing to remove the oligomers and salts. The dialysis tubing was transferred to 3.5% acetic acid solution (˜18 L) and let stand for 7 hours and slowly mix the solution containing the dialysis tubings to complete salt exchange. Then the solution was dialyzed against running de-ionized water for 18-24 hours, collected, filtered through a 0.2-micron filter and lyophilized (freeze dried) to get the solid poly (alanine, glutamic acid, lysine, tyrosine) acetate copolymer.

Yield: 12.44 g (72.0%). Proton NMR showed the complete removal of the ethyl and TFA groups. Measured specific viscosity in water, 1% solution at 25° C. and calculated the viscosity molecular weight (26,200). Also measured GPC MALLS molecular weight (21,300), optical rotation)(−58.6°), and amino acid analysis (alanine:5.90; glutamic acid:2.06; lysine:4.97 and tyrosine:1.07).

### Example 4

Polymerization: 6.667 g (0.057974 mole) of L-alanine NCA, 3.884 g (0.019323 mole) of γ-ethyl-L-glutamic acid NCA, 12.957 g (0.048311 mole) of N^{ε}-TFA-L-lysine NCA and 2.000 g (0.009662 mole) of L-tyrosine NCA were dissolved in 0.45 liter of 1,4-dioxane to make a 0.3M solution. Added ˜2.5 g of charcoal and filtered to get clear colorless solution. The filtered NCA solution was transferred to a 1 liter three neck RB flask equipped with mechanical mixing and a water bath at a temperature of 25-30° C. 4.5 ml of 1N sodium methoxide (0.0045 moles) was placed in 25 ml of 1,4-dioxane. The sodium methoxide solution was added to the NCA solution in one portion with vigorous mixing. The polymerization reaction mixture was mixed for 2 hours and held at 25-30° C. for 18-24 hours.

Precipitation of Protected Polymer: Slowly poured the polymer solution in ˜2,500 ml of DI-water with vigorous mixing. Protected polymer precipitated, mixed for 30 minutes and filtered and washed the polymer with 5×250 ml of DI-water.

Deprotection of Ethyl and TFA Groups: Transferred the wet polymer to a 1,000 ml Erlenmeyer flask. Added 280 ml of 1.5M ethanolic sodium hydroxide and mixed for 0.5 hour. Polymer went into solution within 5 minutes of adding the reagent. Diluted the polymer solution with ˜300 ml of DI-water and mixed for 5 minutes.

Dialysis/Ultra-Filtration and Lyophilization (Freeze Drying): The poly (alanine, glutamic acid, lysine, tyrosine) sodium solution was dialyzed (18-24 hours)/ultra-filtered against running deionized water using ˜12K molecular weight cut off dialysis tubing to remove the oligomers and salts. The dialysis tubing was transferred to 3.5% acetic acid solution (˜18 L) and let stand for 7 hours and slowly mix the solution containing the dialysis tubings to complete salt exchange. Then the solution was dialyzed against running de-ionized water for 18-24 hours, collected, filtered through a 0.2-micron filter and lyophilized (freeze dried) to get the solid poly (alanine, glutamic acid, lysine, tyrosine) acetate copolymer.

Yield: 12.77 g (73.9%). Proton NMR showed the complete removal of the ethyl and TFA groups. Measured specific viscosity in water, 1% solution at 25° C. and calculated the viscosity molecular weight (23,100). Also measured GPC MALLS molecular weight (22,100), optical rotation)(−60.7°), and amino acid analysis (alanine:5.90; glutamic acid:2.10; lysine:5.00 and tyrosine:1.00).

What is claimed is:

1. A method for the production of a polyamino acid random copolymer comprising tyrosine, alanine, glutamic acid, and lysine, the method comprising:
(a) polymerizing a mixture of N-carboxyanhydride of tyrosine, N-carboxyanhydride of alanine, N-carboxyanhydride of R¹ protected glutamic acid, N-carboxyanhydride of a base-labile protected lysine in the presence of a polymerization initiator to form a protected polyamino acid random copolymer; wherein R¹ is an alkyl; and
(b) adding a base to the protected polyamino acid random copolymer of step (a) to form the polyamino acid random copolymer or a pharmaceutically acceptable salt thereof, wherein the base cleaves R¹ from the glutamic acid residue and the base-labile protecting group from the lysine residue.

2. The method of claim 1, wherein the polymerization initiator is a nucleophile.

3. The method of claim 2, wherein the nucleophile is chosen from a metal alkoxide and an amine.

4. The method of claim 1, wherein the molar ratio of the N-carboxyanhydrides to the polymerization initiator in step (a) is from about 5:1 to about 1,000:1.

5. The method of claim 1, wherein the polymerization reaction of step (a) is carried out in the presence of a solvent chosen from dioxane, chloroform, dichloromethane, acetonitrile, and combinations thereof, and the reaction is conducted at a temperature ranging from about 20° C. to about 40° C.

6. The method of claim 1, wherein R¹ is chosen from methyl, ethyl, n-propyl, isopropyl, n-butyl, isobutyl, tert-butyl, straight pentyl, and branched pentyl.

7. The method of claim 1, wherein R¹ is ethyl.

8. The method of claim 1, wherein the base-labile protecting group is chosen from TFA and Fmoc.

9. The method of claim 1, wherein R¹ is ethyl and the base-labile protecting group is TFA.

10. The method of claim 1, wherein the base of step (b) is selected from the group consisting of sodium hydroxide, potassium hydroxide, rubidium hydroxide, and combinations thereof.

11. The method of claim 1, wherein the reaction of step (b) is carried out in the presence of an alcoholic solvent at a temperature ranging from about 15° C. to about 30° C.

12. The method of claim 1, wherein the polyamino acid random copolymer has a mass-average molecular weight of about 2000 to about 100,000.

13. The method of claim 1, wherein the polyamino acid random copolymer has a mass-average molecular weight of about 5000 to about 10,000.

14. The method of claim 1, wherein the ratio of alanine to glutamic acid to lysine to tyrosine present in the polyamino acid random copolymer is about 5:1:4:1 to about 7:3:6:1.

15. The method of claim 1, wherein the ratio of alanine to glutamic acid to lysine to tyrosine present in the polyamino acid random copolymer is about 6:2:5:1.

16. The method of claim 1, wherein the yield of the polyamino acid random copolymer is at least 70%.

17. The method of claim 1, wherein the optical purity of the polyamino acid random copolymer is at least 99%.

18. The method of claim 1, wherein the amino acids comprising the polyamino acid random copolymer are at each occurrence in a D or an L configuration.

19. The method of claim 1, wherein the polyamino acid random copolymer is a pharmaceutically acceptable salt chosen from metal salts, inorganic and organic acid salts.

20. The method of claim 1, wherein the polyamino acid random copolymer is an acetate salt.

21. The method of claim 1, wherein the polyamino acid random copolymer is Copolymer 1.

22. The method of claim 1, wherein the polymerization initiator is a nucleophile chosen from a metal alkoxide and an amine; the molar ratio of the N-carboxyanhydrides to the polymerization initiator in step (a) is from about 5:1 to about 1,000:1; the polymerization reaction of step (a) is carried out in the presence of a solvent chosen from dioxane, chloroform, dichloromethane, acetonitrile, and combinations thereof, and the reaction is conducted at a temperature ranging from about 20° C. to about 40° C.; the base of step (b) is selected from the group consisting of sodium hydroxide, potassium hydroxide, rubidium hydroxide, and combinations thereof; and the reaction of step (b) is carried out in the presence of an alcoholic solvent at a temperature ranging from about 15° C. to about 30° C.
